(19) United States
(12) Patent Application Publication
(10) Pub. No.: US 2009/0062685 A1
(43) Pub. Date: Mar. 5, 2009

(54) ELECTRO-OPTICAL SENSOR FOR PERIPHERAL NERVES

(75) Inventors: Peter R. Bergethon, Dover, MA (US); Sergio Fantini, Winchester, MA (US)

Correspondence Address: RONALD I. EISENSTEIN, 100 SUMMER STREET, NIXON PEABODY LLP, BOSTON, MA, 02110, US

(73) Assignees: TRUSTEES OF BOSTON UNIVERSITY, Boston, MA (US); TRUSTEES OF TUFTS COLLEGE, Medford, MA (US)

(21) Appl. No.: 12/293,146

(22) PCT Filed: Mar. 16, 2007
(86) PCT No.: PCT/US07/06621

§ 371 Date: Sep. 16, 2008

Related U.S. Application Data
(60) Provisional application No. 60/782,841, filed on Mar. 16, 2006.

(51) Int. Cl. A61B 5/00; A61N 1/36

(52) U.S. Cl. 600/554

(57) ABSTRACT

Near-infrared spectroscopy (NIRS) is employed to examine the neuronal activity and vascular response of a peripheral nerve for research or clinical purposes. An embodiment for implementing this approach has: a nerve stimulator; a tissue spectrometer; a stimulation probe adapted to apply a stimulation from the nerve stimulator to a peripheral nerve; at least one illumination optical fiber, where each illumination optical fiber is adapted to transmit a near-infrared source light to the peripheral nerve after the stimulation is applied; and a detection optical fiber adapted to collect and deliver to the tissue spectrometer a returning light from the peripheral nerve after each source light is transmitted to the peripheral nerve. The returning light has a returning intensity, and the tissue spectrometer can determine the returning intensity to provide readings of optical diffuse reflectance of the peripheral nerve after the stimulation is applied.

### CROSS REFERENCE TO RELATED APPLICATIONS

This application claims priority to U.S. Provisional Application No. 60/782,841 filed Mar. 16, 2006, the contents of which are incorporated entirely herein by reference.

### GOVERNMENT SUPPORT

This invention was made with Government Support under Grant No. BES-0093840 awarded by the National Science Foundation. The Government has certain rights in the invention.

### FIELD OF THE INVENTION

The present invention generally relates to analysis of the peripheral nervous system, and more particularly to an apparatus for applying stimulation to a peripheral nerve and measuring the near-infrared optical response of the peripheral nerve to the stimulation.

### DESCRIPTION OF THE RELATED ART

The peripheral nervous system (PNS) is an extensive neural network that connects the central nervous system to the body and its external environment. Diseases and aging affect the PNS causing significant morbidity and mortality, especially when considering that the aging of the peripheral nervous system leads to gait instability and falls. Laboratory evaluation of the peripheral nervous system is largely dependent on electrophysiological testing and the invasive removal of a piece of nervous tissue in a nerve biopsy. These technologies, while effective, have been largely unchanged for 50 years.

The brain and spinal cord, which are contained inside the pial membrane, as well as the optic and olfactory nerves make up the central nervous system. The PNS includes all of the neural structures that lie outside the central nervous system. The peripheral nerves connect the central nervous system to the environment via a sensory (afferent) division and a motor (efferent) division. The sensory system is comprised of cells whose nuclei are located near the spinal cord or brainstem (the dorsal root ganglia or cranial ganglia). The centrally directed axonal processes of these cells project through the spinal cord and brainstem for variable distances before synapsing with secondary neurons in the central nervous system. For instance, pain and temperature fibers synapse in the spinal cord with neurons in the dorsal horns whose axons project to the brain, while the central axons of vibration and proprioceptive fibers travel through the posterior columns of the spinal cord to synapse in the brainstem with their secondary neurons. The peripheral axons of the dorsal root ganglia cells are the sensory nerve fibers and terminate as freely branching or specialized sensory receptors in the joints, skin and other tissues of the body. The peripheral nervous system also includes the motor nerves whose cell bodies lie in the anterior horns of the spinal cord. The axons of the motor nerves join with the peripherally directed sensory axons to form the complex network of peripheral nerves that reach throughout the body.

In addition to the nervous tissue axons, the peripheral nervous system is supported by Schwann cells that produce the fatty sheath of myelin that surrounds many peripheral axons and allows for rapid propagation of the nerve impulses. There is a continuous flow of protein, nucleic acids and metabolic products between the neuron cell bodies and the far-flung axon process supported by an internal system of antegrade and retrograde transport. The function and health of the peripheral nervous system depends greatly on normal axonal transport and on the well being of the myelin-producing Schwann cells. Diseases and aging of the peripheral nervous system can lead to damage of either the myelin or the axons. Aging of the PNS leads to a significant loss of the capacity of an individual to maintain normal balance and gait and manifests itself as falls and unsteadiness in a large proportion of the elderly population.

Nerve conduction studies (NCS) and nerve biopsy are the tools available to the clinical neurologist and the researcher when examining the peripheral nervous system. Measuring nerve conduction velocities as well as sensory and motor action electrophysiological potentials are well-established diagnostic tools that measure the evoked electrical activity of the peripheral nervous system. Yet the electrical activity of the nervous system is often normal even when significant disease processes are affecting the PNS. Thus, methods that reflect metabolic derangements in the PNS would be very valuable.

It is well-established that human tissues are transparent to near-infrared light (650 nm to 1000 nm). Moreover, it has been shown that near-infrared spectroscopy (NIRS) can detect intracranial cerebral hematomas through the skin, scalp, and skull in human subjects. A number of research groups worldwide have recently used NIRS in functional brain studies as a non-invasive tool to monitor local changes in cerebral oxygenation and hemodynamics. The ability to measure certain rapidly changing compounds with absorption in the NIR range such as oxygenated hemoglobin (HbO₂), deoxy-hemoglobin (Hb), and oxidised cytochrome oxidase (CtOx), allows for metabolic information about the nervous system to be obtained in a rapid and non-invasive manner.

Electrophysiological and NIRS signals from the peripheral nerve are biophysically distinct. However, they form a natural complement, because the electrical and optical signals have a close relationship through the physiology of the nervous system. For example, NCS directly measures the electrical activity associated with neuronal excitation, while NIRS is highly sensitive to the hemodynamic response induced by neuronal activation via neurovascular coupling.

Near-infrared light undergoes two physical processes while traveling inside biological tissues. The first process is absorption, described in terms of the linear absorption coefficient (μₐ) that typically assumes values between 0.01 and 1 cm⁻¹ in tissue. Such a low value of μₐ accounts for the large penetration depth, up to several centimeters, of near-infrared light into tissues. Tissue absorption occurs due to the presence of a number of chromophores. In the near-infrared range, the dominant chromophores in tissues are hemoglobin and water, with smaller contributions from lipids and cytochrome oxidase. In particular, with wavelengths between 670 and 850 nm, hemoglobin is the dominant absorber in tissues. This feature renders near-infrared spectroscopy particularly sensitive to changes in the blood flow and blood oxygenation. By taking advantage of the different absorption spectra of oxy-hemoglobin and deoxy-hemoglobin, a measurement of the tissue absorption at two or more wavelengths can be translated into a measurement of the oxygen saturation of hemoglobin. Because of neurovascular coupling, neuronal activation induces changes in the local blood flow that are in turn associated with changes in the local absorption properties of brain tissue.

The second process experienced by photons inside tissues is scattering, which is described in terms of the reduced scattering coefficient (μₛ′). The reduced scattering coefficient is typically two orders of magnitude greater than the absorption coefficient, so that light scattering dominates over absorption in most biological tissues. The strong scattering experienced by near-infrared light inside tissue poses an intrinsic limitation to the spatial resolution of non-invasive optical imaging which is limited to several millimeters. Tissue scattering originates from the discontinuities in the refractive index at the surfaces of cellular membranes and organelles. This feature has suggested the use of the reduced scattering coefficient to non-invasively measure changes in blood glucose concentration. Scattering changes in the brain can relate to changes in the index of refraction of neuronal membranes, in the refractive index mismatch between the intra- and extra-cellular fluids, and to volume changes of cellular compartments. Studies on neuronal cell cultures have suggested that neuronal activation is associated with changes in the optical scattering properties.

Due to the two independent coefficients (absorption and scattering) that characterize the optical properties of tissues, tissue spectroscopy can be performed more effectively by time-resolved methods either in the time-domain (where the light source is pulsed with a pulse width on the order of picoseconds) or in the frequency-domain (where the light source is intensity-modulated at a radio frequency). In fact, the distribution of the photon time-of-flight (in the time-domain) and the amplitude and phase of the photon-density-wave (in the frequency-domain) provide more information than the single parameter (the optical density) provided by continuous-wave (CW) spectroscopy. The additional information content of time-resolved spectroscopy affords the separation of absorption and scattering, and has recently led to absolute tissue oximetry of skeletal muscles and of the brain, as opposed to the relative readings afforded by CW spectroscopy. Furthermore, the different spatial distribution of the region of sensitivity of the various moments of the time-of-flight distribution (time-domain) or of the amplitude and phase (frequency-domain) may provide a more effective approach to the study of brain activity with respect to CW methods.

The relationship between local cerebral hemodynamics and neuronal activation is tied to the concept of neurovascular coupling. This idea is perhaps best appreciated in the brain where local blood flow is continuously adjusted according to the functional activity and metabolic demand. This is achieved by the vasomotor action of the cerebral arteries and arterioles. Both positron emission thomography (PET) and functional magnetic resonance imaging (fMRI), the leading methods in functional neuroimaging, use neurovascular coupling to assess brain activity. PET measures regional cerebral blood flow (rCBF), while fMRI measures blood oxygenation level dependent (BOLD) signals. Both PET and fMRI measure hemodynamics signals having a latency of 3 to 5 seconds, and are therefore only an indirect sign of the fast neuronal activation because electrical signaling in the nervous system occurs on the order of milliseconds.

The NIRS technique uses a probe, i.e., near-infrared radiation, that is potentially sensitive to both ends of the neurovascular coupling, namely the neuronal activity and the late vascular response.

The optical signals associated with brain activation are usually classified into slow signals and fast signals. Slow signals, with a latency of a few seconds, represent the hemodynamic response induced by neuronal activation. Fast signals, with a latency of 10 ms to 100 ms, represent optical signatures more directly associated with neuronal activation. The origin of the fast optical signals is not well understood, as it may be associated with scattering changes that are known to be linked to neuronal activation, as well as absorption changes that may arise from evoked vascular responses.

In vitro optical measurements on neurons have shown that neuronal activity is associated with an increase in light scattering, induced by a change in the index of refraction of the neuronal membranes. In the last few years, several attempts have been made to measure such light scattering changes in human subjects in vivo and non-invasively in the central nervous system. Using a frequency-domain system, a transient increase has been reported in the path-length of light in the visual cortex with a latency of 50 to 100 ms following the onset of visual stimulation. This signal shows a similar time course to the electrophysiological response measured non-simultaneously with EEG. Other groups have recently reported similar results in the somatosensory, visual, and motor cortices, thus confirming the technical feasibility of the non-invasive measurement of the evoked optical fast signal. However, because this optical signal is small (less than 0.1% in the intensity), there are still open questions on the data processing schemes used by the various groups. In particular, it is often necessary to average over a large number (100 to 1000) of repetitions of the stimulus, and to subtract the relatively large (a few percent in the intensity) physiological fluctuations associated with the arterial pulsation.

Near-infrared light penetrates through several centimeters of tissue and has been successfully applied to the non-invasive study of skeletal muscle (for muscle perfusion and oxygenation), breast (for tumor detection), and brain (for functional studies) in human subjects. NIRS is highly sensitive to the concentration and oxygen saturation of hemoglobin in tissue, and therefore it provides physiological information related to the local blood flow, blood volume, oxygen delivery, and metabolic rate of oxygen in tissue. Furthermore, with appropriate amplification and mathematical filtration, NIRS can be sensitive to optical scattering changes that originate at the cellular and organelle level, which have in turn been associated with changes in blood glucose concentration and neuronal activation.

### SUMMARY OF THE INVENTION

Embodiments of the present invention use near-infrared spectroscopy (NIRS) to examine the neuronal activity and vascular response of a peripheral nerve for research or clinical purposes. An embodiment of the present invention provides an apparatus with a nerve stimulator; a tissue spectrometer; a stimulation probe adapted to apply a stimulation from the nerve stimulator to a peripheral nerve; at least one illumination optical fiber, where each illumination optical fiber is adapted to transmit a near-infrared source light to the peripheral nerve after the stimulation is applied; and a detection optical fiber adapted to collect and deliver to the tissue spectrometer a returning light from the peripheral nerve after each source light is transmitted to the peripheral nerve. The returning light has a returning intensity, and the tissue spectrometer can determine the returning intensity to provide readings of optical diffuse reflectance of the peripheral nerve after the stimulation is applied.

In a particular use of the embodiment above, an electrical stimulation probe applies an electrical pulse, with a current between 10 mA to 40 mA, to a sural nerve every 500 ms for 35 seconds. Illumination optical fibers transmit source light with wavelengths of 690 and 830, every 10 milliseconds respectively, to the sural nerve after the electrical stimulation has been applied. The detection optical, separated from the illumination optical fiber by 2 cm, collects the returning light from the peripheral nerve after the source light is transmitted to the peripheral nerve and delivers the returning light to the tissue spectrometer. The tissue spectrometer then measures the intensities of the returning light to enable characterization of the concentration of oxy-hemoglobin and deoxy-hemoglobin resulting from the stimulation of the peripheral nerve.

In another embodiment, the present invention provides an apparatus with a nerve stimulator adapted to apply an electrical stimulation to a peripheral nerve; a tissue spectrometer; a plurality of illumination optical fibers where each illumination optical fiber is adapted to transmit a near-infrared source light to the peripheral nerve after the electrical stimulation is applied; and a detection optical fiber adapted to collect and deliver to the tissue spectrometer a returning light from the peripheral nerve after each source light is transmitted to the peripheral nerve, where a plurality of separation distances separate the plurality of illumination optical fibers from the detection optical fiber. Thus, returning light may be collected for a plurality of distances between the illumination and detection optical fibers, especially to reduce effects from movement artifact.

In an alternative embodiment, the present invention provides a sensing electrode positioned between the illumination optical fibers and the detection optical fiber, where the sensing electrode collects electrical data from the peripheral nerve concurrently with the collection of the returning light by the detection optical fiber. Accordingly, concurrent electrical and NIRS data may be collected to examine how electrophysiological signals correspond to the fast-component near-infrared spectroscopy (NIRS) signature.

Still other aspects, features, and advantages of the present invention are readily apparent from the following detailed description, by illustrating a number of exemplary embodiments and implementations, including the best mode contemplated for carrying out the present invention. The present invention is also capable of other and different embodiments, and its several details can be modified in various respects, all without departing from the spirit and scope of the present invention. Accordingly, the drawings and descriptions are to be regarded as illustrative in nature, and not as restrictive.

### BRIEF DESCRIPTION OF THE FIGURES

FIG. 1 illustrates an exemplary embodiment of the present invention being implemented on a sural nerve.

FIG. 2A illustrates a bottom view of another exemplary embodiment of the present invention employing a plurality of illumination optical fibers, a detection optical fiber, and a sensing electrode positioned on a flat probe.

FIG. 2B illustrates another view of the exemplary embodiment of FIG. 2A.

FIG. 3A illustrates an exemplary set-up for measuring electrical responses for determining the spatial dependence of the optical and electrical responses associated with the electrical stimulation of a peripheral nerve.

FIG. 3B illustrates an exemplary set-up for measuring optical data in association with the set-up of FIG. 3A.

### DETAILED DESCRIPTION OF THE INVENTION

The measurement of the optical changes, particularly fast optical signals, exhibited by the peripheral nerves can have diagnostic value and can open new research opportunities to advance the understanding of the nervous system. Examination of these optical changes provides a new perspective on the functioning and activity of the peripheral nervous system that has not been conceptualized or considered in the prior art, especially with respect to clinical analysis of nerve function or pathology. Measuring these optical responses can permit detailed evaluation of peripheral nerve function and also their diseases (neuropathy). In particular, the capacity to detect vascular changes and relate them to the electrical activity of peripheral nerves provides a completely new measurement in peripheral neurophysiology and in clinical neurophysiology.

Accordingly, embodiments of the present invention provide an electro-optical sensor for the non-invasive study and evaluation of peripheral nerve activity. Such embodiments enable investigation of the optical response of the peripheral nerve associated with electrical nerve stimulation. The chemical biology associated with activity of the peripheral nervous system may be explored, particularly with regard to blood flow correlated to nervous system activity, i.e. neurovascular coupling. Moreover, embodiments of the present invention offer an improved approach for researchers to investigate the optical signatures associated with electrical activity. In addition, embodiments of the present invention have clinical value because they offer new diagnostic tools to investigate peripheral nerve viability and functionality. Uses may include diagnostic methods that measure changes in peripheral nerves associated with: aging especially with respect to gait and balance disorders, common metabolic diseases such as diabetes mellitus, neurotoxins such as anti-neoplastic drugs and alcohol, infectious agents such as HIV and leprosy, artherosclerotic ischemic diseases of the nerves, and autoimmune diseases causing vasculitis.

In one embodiment, the invention enables the physician to establish the normal ranges of fast-component NIRS signal for any specific peripheral nerve or non-peripheral nerve and correlated that data with normal nerve structure and function. The physician can then compare the normal data with data from studies of affected nerves or aging nerves. For example, a loss in the hemodynamic response upon nerve stimulation, and/or a slowing of the fast-component NIRS response may be indicative of compromised nerves or aging nerves. One use of such normal and affected nerve fast-component NIRS signals comparison data is in surgery. Real-time information by way of the fast-component NIRS signal can provide information about regional spinal cord ischemia, and that can guide intraoperative management and reduce the risk of paraplegia after thoracic aortic surgery. Intraoperative spinal cord ischemia is a potential complication faced by patients undergoing thoracic aortic surgery such as heart transplant or a heart by-pass surgery. Scheduled and frequent monitoring of the spinal cord during surgery may be achieved in a heavily sedated patient.

One embodiment of the invention provides for a nerve monitoring tool for use during high-risk procedures such as cranio-facial surgery and thyroid surgery. Nerve monitoring using an apparatus of the invention to monitor motor nerves can reduce the risk of nerve damage during surgery thus helping to protect patient and assist the surgeon. The apparatus is used in these two scenarios to monitor the cranial and spinal peripheral motor nerves during facial surgery and the laryngeal nerve during thyroid surgery, and may also be used in a wide variety of similar high risk procedures to help prevent neural damage.

One embodiment of the invention provides for a clinical diagnostic and monitor device for monitoring the viability and functionality of the peripheral nerves in patients. For example, it can be used clinical indications such as those that affect peripheral blood circulation and the peripheral nervous system: metabolic diseases such as diabetes mellitus; infectious diseases or infections such as HIV infection or leprosy; neurotoxicity resulting from alcohol or cancer therapy such as anti-neoplastic drugs; atherosclerotic ischemia diseases of the nerves; autoimmune diseases such as vasculitis; peripheral neuropathy; neuralgia; Bell's palsy; reflex sympathetic dystrophy syndrome; low-back pain with and without sciatica; Guillain-Barré syndrome, and neuropathic pain. If the fast-component of the NIRS signal is been established prior to disease onset, the physician will have a baseline to compare with during treatment. Timed interval monitoring of the affected peripheral nerve lets the physician know the rate of disease progression as well as the stage of disease in an individual. In addition, monitoring of the affected peripheral nerve during the various appropriate treatment regime, such as corticosteroids and/or immunoglobulin treatment for chronic inflammatory demyelinating polyradiculoneuropathy, provides vital data for the physicians on the efficacy of the treatment.

Another embodiment of the invention provides for screening of new emerging treatments and drugs for the various peripheral nerve diseases and disorders. An apparatus of the invention provide a non-invasive option for monitoring the viability and functionality of periphery nerves in animal models with the respective peripheral neuropathy during the pre-clinical trials of the drugs and/or treatment regime, and then later in human with the respective peripheral neuropathy during clinical trials of the drugs and/or treatment regimes.

Another embodiment of the invention provides for a combined optical imaging of muscles and nerves. This permits, for example, the examination of the neuromuscular junction by a non-invasive approach. The combined optical imaging apparatus will have nerve stimulating electrodes, illumination optical fibers emitting the NIRS signal, detection optical electrodes for the NIRS signal, and detection electrodes for detecting mechanoactivity such as muscle twitching/movement. This provides data showing the relationships between nerve physiological structure (thus its physiological health), nerve stimulation and strength, and muscle contraction and strength. Studies of the neuromuscular junction are particularly important for exercise physiology, clinical neurology, and sports training of professional performance athletes.

In one embodiment, the invention provides for the molecular imaging of peripheral nerves using NIRS-sensitive fluorophores. The molecular and structural visualization of the physiological components of nerve cells can be achieved by way of fluorescent chromophores that are taken up by neurons and thus are used to label the neuron cell body, dendrites, and axonal processes. Characteristics of a good NIRS-sensitive fluorescent chromophore include (1) the ability to be taken up into the axon and transported; (2) diffuse along and across cell membranes; (3) absorb and emit fluorescence at NIR wavelengths (preferably 700-800 nm) or absorb at NIR wavelengths; and (4) have low autofluorescence. There are several commercially available NIR fluorescent chromophores including IRDye® 800CW, IRDye® 680, IRDye® 700DX, Cy5.5, Alexa® Fluor 750, Alexa® Fluor 680, FluoSpheres Far Red, FluoSpheres Infar red, 1,1′-dioctadecyl-3,3,3′,3′-tetramethylinotricarbocyanine iodide (di-R), the various lipophilic indocarbocyanine dyes (DiI, DiA, DiD, DiO, PKH2, PKH26), and indocyanine green. The indocyanine green is the preferred choice as it is current being used to clinically. Depending on the water solubility of these dyes, the dyes may be mixed with a solvent such as DMSO or packaged in liposomes and introduced by known means such as topically on the skin or intravenously or intrathecally into a subject such as a human. With time the dye, depending the type used, will become covalently attached to the neuronal membrane, taken up by the neurons into lysosomes, and/, or transported along the microtubules of the processes. Alternatively, these dyes may be conjugated to other molecules to specifically target a compartment within the neuron, for example, dye-anti-NMDA anti-body conjugate will target the dendritic synapses where NMDA receptors are concentrated. Dye-Protein Conjugation kits are commercially available as well. Appropriate highpass filters can be incorporated into the photomultiplier to measure the fluorescent signal from these chromophores. The illumination from the NIR laser diode (illumination fiber 242) will be at a suitable wavelength (which is dependent on the absorption wavelength of the NIR-sensitive chromophore), while the fluorescence emission will be isolated with a long-pass filter at the tip of the detection fiber 244.

Embodied in the invention is the use of molecular imaging approach to study the structural and functional changes of nerve cells and axons during aging. With age, it is possible that structural changes can lead to functional changes, and these functional changes may be represented as a loss in the hemodynamic response upon nerve stimulation, and/or a slowing of the fast-component NIRS response observed in younger subjects.

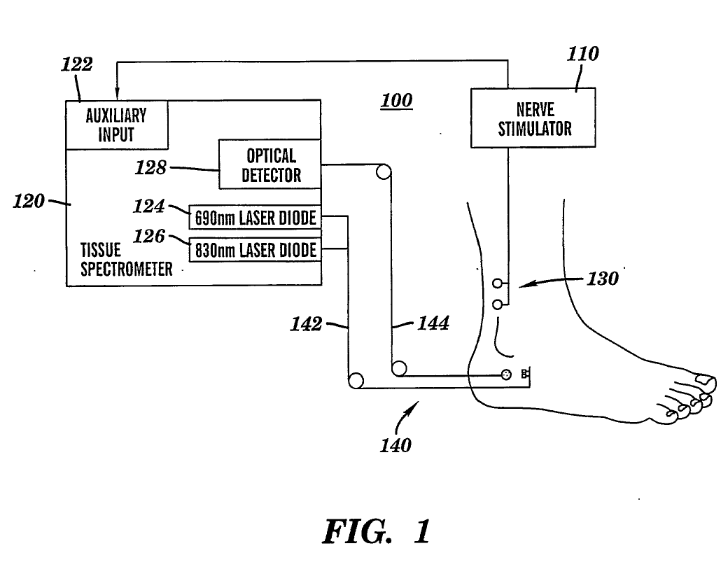

An embodiment of the present invention is illustrated in FIG. 1. In order to stimulate a peripheral nerve and detect the optical response associated with the electrical stimulation, the electro-optical assembly 100, as shown in FIG. 1, employs an electrical nerve stimulator 110 and a near-infrared tissue spectrometer 120. An example of an electrical nerve stimulator usable with the electro-optical sensor 100 is the Nicolet Biomedical Viking Select electromyography (EMG)/nerve conduction study (NCS) machine from VIASYS Healthcare Inc. Neurocare Group (Madison, Wis.). An example of a near-infrared tissue spectrometer usable with the electro-optical sensor 100 is the OxyplexTS from ISS, Inc. (Champaign, Ill.).

As shown in FIG. 1, the electrical nerve stimulator 110 has an electrical stimulation probe 130, which is placed on the skin to excite a peripheral nerve with electrical stimulation. For instance, the electrical stimulation probe 130 may be coupled to the skin over the peripheral nerve with conducting gel and secured with tape. The near-infrared tissue spectrometer 120 uses near infrared light to optically detect the effects of this nerve stimulation.

The tissue spectrometer 120 is connected to optical fibers 140, which include at least one illumination, or source, optical fiber 142 and a detection optical fiber 144. The illumination fiber 142 transmits near-infrared light to the tissue, and the detection optical fiber 144 collects the light returning through the tissue. The illumination fiber 142 and the detection optical fiber 144 may be optically coupled to the skin via prisms, which can deflect the light to/from a direction perpendicular to the optical fibers. In this way, the optical fibers 130 may be oriented parallel to the skin. Moreover, due to the internal reflection within the prisms, the diameter of the illumination spot size on the skin may be larger than the diameter of the diameter of the illumination fiber 142. For example, while the illumination fiber 142 may be 400 μm in diameter, the prisms may create a spot that is approximately 2 mm.

The detection optical fiber 144 is connected to the tissue spectrometer 120 through an optical detector 128, which may be a photomultiplier tube (PMT) detector. The intensity of the returning light is determined by the tissue spectrometer 120 to provide readings of the optical diffuse reflectance of the tissue responding to the electrical stimulation.

In an exemplary use of the present invention, as illustrated in FIG. 1, the electrical stimulation probe 130 is placed along the sural nerve, about 5 cm proximate to the ankle. It is understood, however, that FIG. 1 only illustrates an exemplary use of the present invention and in no way limits the present invention to use on a particular peripheral nerve, such as the sural nerve.

The optical fibers 140 are placed on the location so that the location of maximum electrical response for the peripheral nerve is between the illumination fiber 142 and the detection fiber 144. The optical fibers 140 may be secured into position with tape and then wrapped by a black elastic band to shield any outside light from affecting the measurements. Before positioning the optical fibers 140, a recording electrode may be placed on different places about the peripheral nerve to find the location of the maximum electrical response while the nerve is being stimulated.

Moreover, the level of current applied to the nerve may be initially varied to determine the threshold for motion in the area where the optical fibers 140 are to be placed. To avoid potential artifacts and effects associated with tissue motion and related effects from probe-to-skin contact, measurements are taken below a threshold of visible tissue twitching resulting from the stimulation. One possible way to establish this threshold is to place a mirror on the tissue surface to be examined and to reflect a laser beam off the mirror onto a wall, or surface, some distance, e.g. 3 m, away to amplify the angular magnitude of the twitching effect. The threshold is the current that is high enough to stimulate twitching that in turn causes the reflected laser to move on the wall or surface.

In the example illustrated in FIG. 1, the illumination fiber 110 and the detection fiber 120 are separated by a source-detector distance equal to about 2 cm, and are placed so that the nerve is between the illumination fiber 110 and the detection fiber 120. Each measurement consists of a 5 to 30 second baseline where intensity is measured with no electrical stimulation. This is then followed by a 35 second period of nerve stimulation in the form of a train of 1 ms pulses every 500 ms. Current levels are within the range of 10 mA to 40 mA, depending on the threshold for twitching motion.

In order to synchronize the stimulus with the collection of optical data, an external output signal is sent from the electrical nerve stimulator 110 to an auxiliary input channel 122 of the tissue spectrometer 120 when the electrical stimulation period is started. The tissue spectrometer 120 provides readings of the optical diffuse reflectance of tissue at two near-infrared wavelengths of 690 nm and 830 nm after stimulation in order to make determinations for oxy-hemoglobin and deoxy-hemoglobin. The illumination fiber 142 may actually be a pair of closely-spaced fibers, with one fiber connected to a 690 nm laser diode 124 to guide light at 690 nm and the other fiber connected to a 830 nm laser diode 126 to guide light at 830 nm. The optical sampling frequency is 50 Hz, sequential data acquisition of 10 ms at the two wavelengths. A temporal resolution of 20 ms or better may be needed for the optical data, to follow the fast electrical dynamics associated with nerve stimulation.

Although the exemplary uses described herein employ specific parameters, these parameters are provided only as an example, and in no way, do they limit how the present invention is implemented. For instance, the frequency of stimulation is not limited to the application of a pulse every 500 ms. In addition, embodiments of the permit the use of the full spectra in the near-infrared range, 650-1000 nm, and is not limited to the use of the two wavelengths of 690 nm and 830 nm. Moreover, the distance between the illumination fiber 142 and the detection fiber 144 is not limited to 2 cm, but might have a greater range, e.g. 1 to 2 cm for the sural nerve. Indeed, measurements at a second source-detector distance may help rule out effects associated with movement and contact artifact. To permit measurements of different source-detector distances, an alternative embodiment of the present invention arranges the illumination and detector fibers in a flat optical probe with one detector fiber and multiple illumination fibers embedded along the probe. Each illumination fiber forms a pair with the detector fiber, and each pair has a different source-detector distance.

To analyze the intensity data collected by the near-infrared tissue spectrometer 120, a folding average procedure is applied, in which the relative intensity changes observed in each 500 ms period between successive stimulations are averaged. The modified Beer-Lambert Law is applied to the data to translate the intensity traces into traces of the concentrations oxy-hemoglobin and deoxy-hemoglobin in the analyzed tissue. The results generally show that the optical intensity has a different behavior at two wavelengths. The oxy-hemoglobin and deoxy-hemoglobin concentrations calculated from the modified Beer-Lambert law follow a similar divergence with the deoxy-hemoglobin concentration ([Hb]) increasing and the oxy-hemoglobin concentration ([HbO₂]) decreasing in response to the electrical stimulus. For instance, in an exemplary trial the intensity at 830 nm increases to a maximum around 60 ms after stimulation and returns to baseline around 180 ms after the stimulus. Meanwhile, the intensity at 690 nm increases slightly for about the first 20 ms after stimulation before decreasing to a minimum at about 100 ms after stimulation. Each response to the stimulation lasts approximately 300 ms and peaks within less than 100 ms. This occurs regardless of the frequency of stimulation. The net total effect is a decrease in the total hemoglobin concentration ([HbT]) in response to the stimulus.

Such results are consistent with a vascular response associated with the electrical stimulation. The fact that the optical response at the two wavelengths is qualitatively different (intensity increase at 690 nm, decrease at 830 nm) rules out the possibility that this response results from a motion artifact, and makes it unlikely to be originated by scattering changes. Where hemoglobin is the origin of this fast optical signature, the results are consistent with a transient increase in oxygen saturation of hemoglobin, a result of a transient increase in blood flow. The response to stimulation with a decrease in deoxy-hemoglobin concentration ([Hb]) and an increase in oxy-hemoglobin concentration ([HbO₂]) is the signature for an increase in blood flow.

The basic approach to the analysis of data measured for one source-detector distance is to translate the temporal changes in the intensity DC(t) (with respect to the initial intensity DC(0)) into changes in the tissue absorption coefficient (Δμₐ) using the differential-pathlength-factor (DPF) method:

$${Δμ}_{a} ( t ) = \frac{1}{r DPF} ln [ \frac{DC ( 0 )}{DC ( t )} ] , \qquad ( 1 )$$

where r is the source-detector separation. The value of the DPF at two wavelengths λ₁ and λ₂ can be measured for each subject using frequency-domain data, or can be assumed on the basis of literature data. From the values of Δμₐ^{λ1}, and Δμₐ^{λ2} one can obtain the temporal changes in the cerebral oxy-hemoglobin (Δ[HbO₂]) and deoxy-hemoglobin (Δ[Hb]) concentrations according to the following equations:

$$\begin{matrix}Δ [ {HbO}_{2} ] = \frac{{Δμ}_{a}^{λ 1} {ɛ}_{Hb}^{λ 2} - {Δμ}_{a}^{λ 2} {ɛ}_{Hb}^{λ 1}}{{ɛ}_{HbO 2}^{λ 1} {ɛ}_{Hb}^{λ 2} - {ɛ}_{Hb}^{λ 1} {ɛ}_{HbO 2}^{λ 2}} , & ( 2 ) \\ Δ [ Hb ] = \frac{{Δμ}_{a}^{λ 2} {ɛ}_{HbO 2}^{λ 1} - {Δμ}_{a}^{λ 1} {ɛ}_{HbO 2}^{λ 2}}{{ɛ}_{HbO 2}^{λ 1} {ɛ}_{Hb}^{λ 2} - {ɛ}_{Hb}^{λ 1} {ɛ}_{HbO 2}^{λ 2}} , & ( 3 )\end{matrix}$$

where ε indicates the known molar extinction coefficient of Hb or HbO₂ (according to the subscript) at λ₁ or λ₂ (according to the superscript). Although this approach to measuring changes in the oxy- and deoxy-hemoglobin concentrations relies on tissue homogeneity, it is believed that its relative results, i.e. the direction of changes, are reliable.

One may follow the electrophysiological signals and the fast-component near-infrared spectroscopy (NIRS) signature as the nerve impulses travel along the axon. The coordination of electrical impulse propagation and fast hemodynamic response may provide information of clinical and physiological significance.

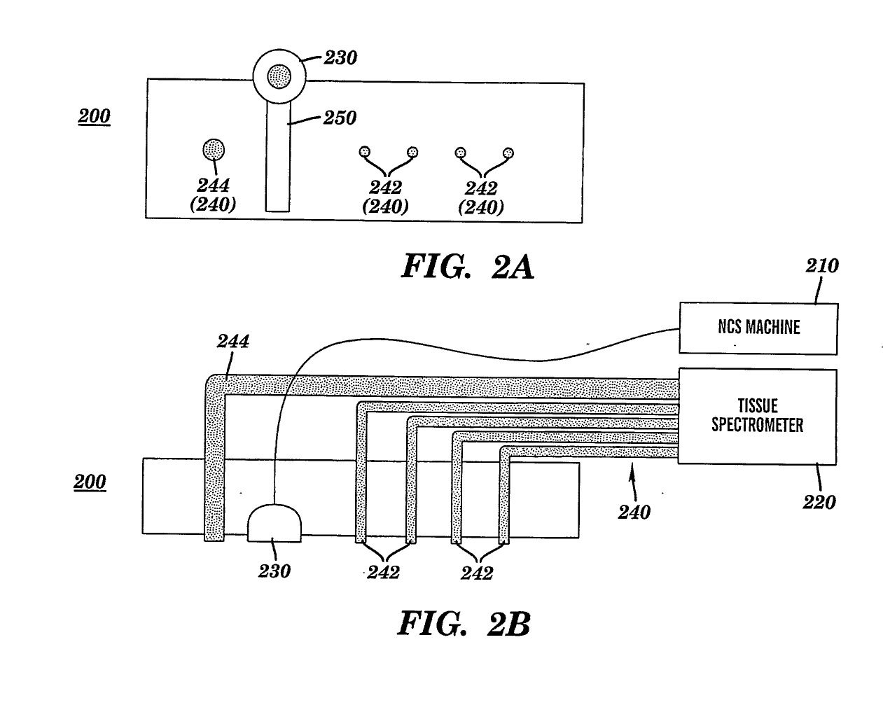

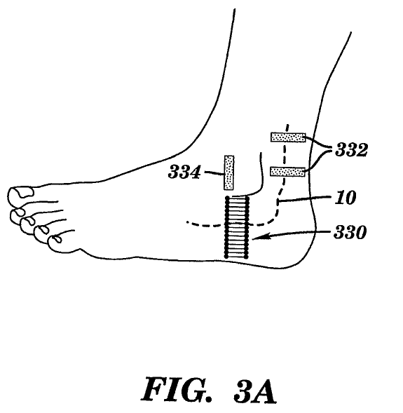

In response to the need for technology that must record the temporal and spatial interactions, another embodiment of the present invention, illustrated in FIGS. 2A and 2B, shows an electro-optical sensor 200, which combines a small NCS sensing electrode 230 with NIRS optical fibers 240 for the simultaneous collection of localized nerve action potentials and near infrared light. A number of electro-optical sensors 200 may be arranged into a series of flexible bands that may be applied to the subject so that a “neuro-electrovascular” image of the peripheral nerve may be determined.

As further illustrated in FIGS. 2A and 2B, the optical fibers 240, connected to the tissue spectrometer 220, include illumination fibers 242, which transmit near-infrared light to the tissue, and detection fiber 244, which detects the optical response of the tissue. The detection optical fiber 244 is a fiber bundle with an internal diameter of 2 mm to 3 mm to maximize the collected optical signal. The illumination optical fibers 242 are also fiber bundles with an active diameter of 1 mm, which are bifurcated and SMA-terminated at the other end for coupling to laser diodes of the tissue spectrometer 220. The bifurcation allows each illumination fiber to transmit two wavelengths, i.e. 690 and 830 nm, for the measurements of oxy-hemoglobin and deoxy-hemoglobin.

For each set of NIRS optical fibers 240, the NCS electrode 230 is placed at the same location. The idea is to collect concurrent electrical and NIRS data, where “concurrent” refers to both temporal and spatial coordinates. Given the spatial dimensions of the peripheral nerve in many cases, the co-localization of the NCS sensing electrode 230 and the NIRS optical fibers 240 means that the NCS and NIRS data will be derived from the same portion of the nervous system. Thus, the electro-optical sensor 200 determines how the electrical activity is locally coupled to the hemodynamic changes sensed by the NIRS fast neuronal activation signal. Accordingly, as shown in FIG. 2A, the NCS sensing electrode 230 and near infrared spectroscopy (NIRS) optical fibers 240 are positioned in proximity on the electro-optical sensor 200. In particular, the sensing electrode is positioned between the illumination fibers 242 and the detection fiber 244. A black light block 250 serves the purpose of preventing light that has not traveled inside the tissue from reaching the detection fiber 244. Preferably, the light block 250 is made of a soft black material.

The NIRS optical fibers 240 includes a plurality of illumination fibers 242 and one detection fiber 244. Each illumination fiber 242 forms a source-detector pair with the single detection fiber 244, with each pair having a different source-detector distance. Each optical fiber is bent by 90 degrees, as shown in FIG. 2B to guarantee that the fiber cables are parallel to the tissue surface, making it easier to secure the electro-optical sensor 200 to the tissue. Alternatively, light from the illumination fibers 242 and the detection optical fiber 244 may be redirected by 90 degrees through the use of prisms, so that optical fibers 240 may generally be oriented parallel to the skin.

Thus, the NIRS data may be collected at multiple separations between the illumination fibers 242 and the detection optical fiber 244. In other words, optical data corresponding to different distances between the illumination and collection optical fibers is collected in the period between electrical stimulations. In contrast to a method collecting data from only one distance, the main advantage of this multi-distance approach is that it is much less sensitive to motion artifacts or to changes in the optical coupling between the electro-optical sensor and the tissue. The advantages of the multi-distance approach is critical, because the tolerance to motion artifact is essential to the use of the electro-optical sensor in real-world situations for evaluating the peripheral nervous system. However, the use of additional light sources in an approach based on electronic multiplexing of the light sources comes at the expense of temporal resolution. Typically, using acquisition times of 10 ms per light source results in a temporal resolution of 20 ms when a two-wavelength, single-distance approach is used. On the other hand, a temporal resolution of 40 ms results when a two-wavelength, two-distance approach is used with the same acquisition times. With an excellent signal-to-noise ratio for the measurement, collecting data every 40 ms may still be meaningful. If a temporal resolution of 20 ms is necessary, however, the single-distance approach may be implemented, and data is collected at two wavelengths to assess the influence of motion artifact, which should contribute equally at the two wavelengths. Indeed, use of the electro-optical sensor 200 does not necessarily require more than one source-detector distance, though multiple separations are available in its design.

The multi-distance electro-optical sensor 200, illustrated in FIG. 2A, features an optimal source-detector distance range where each source-collector pair achieves a sufficient optical penetration depth to probe the peripheral nerve, so that the multi-distance data will most effectively cancel the contributions from skin and muscle tissue. In the case of sural nerve, the distances between the source-collector pairs may range from 1.0 cm to 4.0 cm, though the preferred range is 1.0 cm to 2.0 cm. The data from the source-collector pair with the greater distances, e.g. 4.0 cm, may be disregarded if the signal-to-noise ratio associated with this source is not adequate. However, the source-collector distances discussed herein are provided only as examples, as the source-collector distances may vary according to the nerve being examined and its location relative to the skin surface.

The analysis of multi-distance data is based on a theoretical treatment using diffusion theory. Diffusion theory may be used to specify the dependence of the amplitude (ac), and the phase (Φ) of the modulated intensity on the source-detector separation (r). In an infinite geometry, the ln(r²×ac) and the phase are linear functions of r. In a semi-infinite geometry, where the optical fibers are placed on the interface between a scattering medium and a non-scattering medium, to a first approximation the ln(r²×ac) and Φ are a linear function of r. The semi-infinite case is used as it is a better model for near-infrared spectroscopy of tissue. If we denote with Sac and SD, respectively, the slopes of ln(r²×ac) and Φ as a function of r, the absolute values of μₐ and μₛ′ of a semi-infinite medium in the diffusion approximation are given by:

$$\begin{matrix}{μ}_{a} = \frac{ω}{2 v} ( \frac{{S}_{Φ}}{{S}_{ac}} - \frac{{S}_{ac}}{{S}_{Φ}} ) , & ( 3 ) \\ {μ}_{s}^{′} = \frac{{S}_{ac}^{2} - {S}_{Φ}^{2}}{3 {μ}_{a}} - {μ}_{a} , & ( 4 )\end{matrix}$$

where ω is the angular modulation frequency of the source intensity, and ν is the speed of light in the tissue. The slope of ln(r²×ac) approximates the slope of a more complicated analytical function of r, ac, μₐ and μₛ′. This is an excellent approximation when r\sqrt{3μₐμₛ′}>>1. Equations (3) and (4) show that the multi-distance, frequency-domain method achieves quantitative measurements of both absorption and reduced scattering coefficient. This has been confirmed experimentally in homogeneous, tissue-like materials. However, because of the inhomogeneity of tissues and because of the necessarily approximate treatment of the boundary conditions, some additional empirical measurements may preferably be done. This may readily be done by the skilled artisan. In particular, the separation of absorption and scattering may not always be complete. However, it has been found that the non-invasive measurements of μₐ, and μₛ′ in some experiments contains independent information, so that the separation of absorption and scattering is at least partially accomplished.

A quantitative measurement of the tissue absorption coefficient at two near-infrared wavelengths λ₁ and λ₂, can be translated into a measurement of the concentrations of oxy-hemoglobin [HbO₂] and deoxy-hemoglobin [Hb] under the assumption that hemoglobin is the dominant absorber in tissue at the wavelengths used. In the near-infrared, this assumption is usually satisfied. By indicating with ε the molar extinction coefficient of Hb or HbO₂ (according to the subscript) at λ₁ or λ₂ (according to the superscript), [HbO₂] and [Hb] are given by:

$$\begin{matrix}[ {HbO}_{2} ] = \frac{{μ}_{a}^{λ 1} {ɛ}_{Hb}^{λ 2} - {μ}_{a}^{λ2} {ɛ}_{Hb}^{λ 1}}{{ɛ}_{HbO 2}^{λ 1} {ɛ}_{Hb}^{λ 2} - {ɛ}_{Hb}^{λ 1} {ɛ}_{HbO 2}^{λ 2}} , & ( 5 ) \\ [ Hb ] = \frac{{μ}_{a}^{λ 2} {ɛ}_{HbO 2}^{λ 1} - {μ}_{a}^{λ1} {ɛ}_{HbO 2}^{λ 2}}{{ɛ}_{HbO 2}^{λ 1} {ɛ}_{Hb}^{λ 2} - {ɛ}_{Hb}^{λ 1} {ɛ}_{HbO 2}^{λ 2}} , & ( 6 )\end{matrix}$$

From [HbO₂] and [Hb], one can readily derive the total hemoglobin concentration ([HbT]=[HbO₂]+[Hb]), and the oxygen saturation of hemoglobin in tissue (StO=[HbO₂]/[HbT]. These measurements are absolute but their accuracy may be affected by the inhomogeneity of the tissues that we are investigating. The added value of phase measurements in frequency-domain spectroscopy lies in the (at least partial) separation of absorption and scattering contributions to the diffusely reflected signal.

Alternatively, multi-distance data may be used to find relative absorption changes under the assumption that the reduced scattering coefficient (μₛ′) is constant. The relationship between the spatial slope of the intensity (S_{dc}) and the absorption change is Δμₐ=2S_{dc}ΔS_{dc}/(3μₛ′). This results in an improved signal-to-noise ratio at the expense of the additional information content provided by the phase data, but keeping the insensitivity to motion artifacts featured by multi-distance data.

After translating the optical data into optical coefficients (absorption, scattering) or into concentrations of oxy- and deoxy-hemoglobin, a folding average is performed over the period of stimulation. The fast optical signal that is expected in peripheral nerves appears to be much larger than that reported in the brain. However, band-pass filters, which are routinely applied in brain studies, could be employed to suppress the effects of arterial pulsation in the optical data. It is also noted that multi-distance data also significantly reduces the pulsatile component of the optical data at the heart rate.

The electro-optical sensor 200, illustrated in FIGS. 2A and 2B, employs two principles: placing the NCS electrode 230 and the NIRS fibers 240 at the same location and collecting NIRS data at multiple source-detector separations. However, embodiments of the present invention do not have to implement these two principles in combination. In other words, the NCS electrode and the NIRS fibers may positioned at the same location, without having more than on source-detector separation. Conversely, more than one source-detector separation may be available on one sensor, but the electrode may be placed at another location.

While the exemplary embodiments discussed herein employ electrical stimulation to stimulate the peripheral nerve, embodiments of the present invention also extend to other ways of stimulation, including, but not limited to, mechanical or vibratory stimulation. The use of vibration may provide better simulate the types of stimulation encountered by a peripheral nerve in everyday situations. Moreover, the vascular response may be measured in relation to the varying aspects of vibration, such as amplitude, frequency, and phase.

Further research on the hemodynamic nature of the fast-response signature in the peripheral nerve is facilitated by embodiments of the present invention. In particular, embodiments of the present invention enable peripheral nerve responses to be examined over the full spectra in the NIR range. In addition, the spatial and temporal course of peripheral nerve stimulation may be examined by having multiple sensors placed along the nerve in order to follow the normal relationship between the activation of the nerve and the fast-signal generated in an increasingly distal spatial and temporal dimension following stimulation.

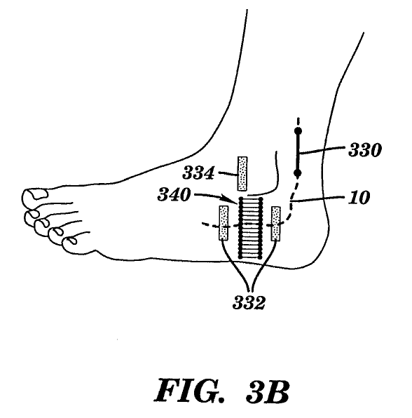

FIGS. 3A and 3B illustrate an exemplary technique for determining the spatial dependence of the optical and electrical responses associated with the electrical stimulation of a peripheral nerve, such as the sural nerve 10. FIG. 3A illustrates a set-up for electrical data collection while FIG. 3B illustrates a set-up for optical data collection according to an embodiment of the present invention. As described previously, the stimulation electrode, or probe, 330 provides an electrical stimulation at a current that is below the threshold of any visible motion to avoid motion-related artifacts in the optical data. For example, an electrical stimulation of 0.1 ms at a frequency of 1.5 Hz may be applied and a current between 10 mA and 40 mA. The stimulation electrode 330 is coupled to the skin over the sural nerve 10, as shown in FIGS. 3A and 3B, with conducting gel and is secured with tape. Recording electrodes 332 are placed distal to the stimulation electrode 330 when recording data. A reference electrode 334 is placed on the skin over the lateral malleolus and used to subtract the common signal of the unrelated tissue from the differential signal of the two recording electrodes 332.

The location of the sural nerve 10 is identified by the position of the largest sensory nerve action potential (SNAP) measured by an EMG monitor and traced distally along the nerve. Spaced recording positions are marked. For example, 16 different positions separated by 2 mm may be marked, stretching from the bottom of the lateral malleolus to the sole of the foot (range from 0 to 30 mm). Due to electrode geometries, the positions of the recording electrode 332 and stimulation electrode 330 in FIG. 3A may be switched from their positions in FIG. 3B in order to record the electrical responses spatially. Thus, for electrical data, the stimulation electrode 330 is placed at each previously marked position and SNAPs are recorded at each position. FIG. 3A illustrates the positions of the stimulation electrode 330.

An optical spectrometer, such as those described previously, is used for the NIRS measurements. In particular, the optical spectrometer may feature one photomultiplier tube detector and two fiber-coupled laser diodes emitting at 690 and 830 nm. The optical probe 340 includes a detection optical fiber bundle and two illumination source fibers. Prisms may be employed to direct the light to/from the optical probe 340, depending on the positioning and orientation of the optical probe 340. As discussed previously, the distance between the illumination fiber and detection optical fiber fibers may be 1.5 cm.

After obtaining electrical data, recording electrodes 332 and the stimulation electrode 330 are repositioned as shown in FIG. 3B. Optical data may then be collected at the same previously marked positions. FIG. 3B illustrates the positions of the optical probe 340. Synchronization between the electrical stimulation and the NIRS instrument is provided by an auxiliary input channel in the NIRS instrument. Data may be acquired at a frequency of 50 Hz, corresponding to an acquisition time of 20 ms per data point. Each trial may last about 30 seconds, during which about 45 electrical pulses are administered. The optical probe position is moved to another marked position after each trial to collect data from all positions. The trials may be repeated several times, e.g. five times, through the entire set of marked positions. A folding average over a 600 ms period may be applied to the optical intensity data over all of the 45 stimulating pulses. The changes in intensity of the trials are averaged and the maximum change at each position is recorded. As discussed previously, using the modified Beer-Lambert law, the optical data, expressed in terms of relative change in intensity, can be translated into changes in concentrations of oxy-hemoglobin [HbO], deoxy-hemoglobin [Hb] and total hemoglobin [HbT].

Once the data has been collected according to the technique illustrated by FIGS. 3A and 3B, the electrical measurements indicate the electrical response for the range of positions of the stimulation electrode 330. The optical data provides the relative intensity changes for the range of positions of the optical probe 340. Some positions may not have a measured electrical signal or relative intensity change. The electrical and optical data can then be compared and analyzed. For example, in a study implementing the set-up of FIGS. 3A and 3B, an electrical response to the stimulating pulse may be detected at coordinates 6-14 mm across the nerve, and a maximum electrical response may be recorded at 12 mm. Meanwhile, relative intensity changes may be present at coordinates 8-16 mm, with a maximum average change in intensity recorded at 14 mm. Such results would suggest that the lateral spatial extent of both signal types is similar, i.e. 8 mm.

Embodiments of the present invention also provide researchers a way to develop a system that can image the peripheral nerve in both the aging and neurologically ill population. The ability to image these interactions along the length of the nerve may reveal potential mechanisms that may contribute to an understanding of the length dependent pattern seen in many neuropathies. In particular, as a spectrophotometric technique, NIRS has the capacity to interrogate not only intrinsic biochemical species but can be sensitive to molecular species added to living tissue therefore giving it a capacity for biological imaging of molecular markers. NIRS has the capacity to enable monitoring of optically active fluorochromes to be able to reflect the metabolic and structural integrity of the peripheral nerve in vivo. There are a variety of fluorescent molecules and photon absorbing dyes that may be used to label neurons and the axonal processes.

Two primary factors determine the utility of a molecular imaging agent, 1) its capacity for being taken up or infiltrating the neuronal structure of interest, and 2) the optical wavelengths at which animal tissues are transparent (650-1000 nm). The choices of potential fluorochromes useful for peripheral nerve molecular imaging operate under these constraints. As a NIR chromophore, indocyanine green may be preferred. It is widely used in both clinical and research applications and is FDA approved for administration to humans. Though it is usually used for angiography and cardiac output measurements and is valued because it remains confined to the vascular compartment when administered intravenously, it has been demonstrated that indocyanine green can undergo both antegrade and retrograde axonal transport in neurons. A significant advantage of indocyanine green is that its maximum absorption occurs at 788 nm that is near the isobestic point of the hemoglobin-oxyhemoglobin pair. Thus, it does not interfere with evaluation of oxygen delivery and consumption in the imaging of the peripheral nerves through NIRS interrogation of this important intrinsic chromophore. However, other chromophores with absorbance and fluorescence in the NIR spectral range are readily available commercially. Each may be used as a dye that increases absorption once it has entered the nerve and can be detected by the NIRS system. In addition with an appropriate highpass filter on the photomultiplier, the fluorescence signal may be measured for each as well.

The finding of a rapid hemodynamic response by the peripheral nerve to stimulation has important clinical implications. The possibility that there is a rapid metabolic response to stimulation is suggestive that disease or the aging states in which the capacity for such a normative response is lost may contribute to clinically relevant pathology is very important. In diabetic neuropathy and cases of vascular insufficiency either due to atherosclerotic or inflammatory vasculitic causes, the suggestion of a derangement in hemodynamics to a functioning nerve may help explain the distal to proximal pattern of disease, provide a new way to diagnose and evaluate treatment course in the peripheral neuropathies and suggest new interventions for the prevention and acute treatment of neuropathies.

While the present invention has been described in connection with a number of exemplary embodiments, and implementations, the present inventions are not so limited, but rather cover various modifications, and equivalent arrangements, which fall within the purview of prospective claims.

1. An apparatus for examining a peripheral nerve, said apparatus comprising:
a nerve stimulator;
a tissue spectrometer;
a stimulation probe adapted to apply a stimulation from the nerve stimulator to a peripheral nerve;
at least one illumination optical fiber, each illumination optical fiber adapted to transmit a source light, having a near-infrared wavelength, from the tissue spectrometer to the peripheral nerve after the stimulation is applied to the peripheral nerve; and
a detection optical fiber adapted to collect and deliver to the tissue spectrometer a returning light from the peripheral nerve after each source light is transmitted to the peripheral nerve.

2. The apparatus according to claim 1, wherein the nerve stimulator is an electrical nerve stimulator and the stimulation current applied is below a threshold of visible tissue twitching.

3. The apparatus according to claim 1, wherein the returning light has a returning intensity and the tissue spectrometer determines the returning intensity to provide readings of optical diffuse reflectance of the peripheral nerve after the stimulation is applied to the peripheral nerve.

4. The apparatus according to claim 1, wherein at least one illumination optical fiber and the detection optical fiber are positioned on opposite sides of the peripheral nerve.

5. The apparatus according to claim 1, wherein at least one illumination optical fiber and the detection optical fiber are positioned on a flat optical probe.

6. The apparatus according to claim 1, wherein the wavelength of the source light transmitted by each of the at least one illumination fiber is about 690 nanometers or about 830 nanometers.

7. The apparatus according to claim 1, wherein each of the at least one illumination optical fiber transmits the source light at least every 20 milliseconds for a period of time.

8. The apparatus according to claim 1, wherein the nerve stimulator sends a synchronization signal to the tissue spectrometer when the stimulation is applied to the peripheral nerve.

9. The apparatus according to claim 2, wherein the electrical stimulation from the electrical nerve stimulator is applied with a current from 10 to 40 milliamperes.

10. The apparatus according to claim 1, wherein the at least one illumination optical fiber and the detection optical fiber are separated by a distance of 1 to 2 centimeters.

11. An apparatus for examining a peripheral nerve, said apparatus comprising:
a nerve stimulator adapted to apply an electrical stimulation to a peripheral nerve;
a tissue spectrometer;
a plurality of illumination optical fibers, each illumination optical fiber adapted to transmit a source light, having a near-infrared wavelength, from the tissue spectrometer to the peripheral nerve after the electrical stimulation is applied to the peripheral nerve; and
a detection optical fiber adapted to collect and deliver to the tissue spectrometer a returning light from the peripheral nerve after each source light is transmitted to the peripheral nerve,
wherein a plurality of separation distances separate the plurality of illumination optical fibers from the detection optical fiber.

12. The apparatus according to claim 11, further comprising a sensing electrode positioned between the plurality of illumination optical fibers and the detection optical fiber, the sensing electrode adapted to collect electrical data from the peripheral nerve after the electrical stimulation is applied to the peripheral nerve.

13. The apparatus according to claim 12, wherein the sensing electrode collects the electrical data concurrently with the collection of the returning light by the detection optical fiber.

14. The apparatus according to claim 12, wherein the plurality of illumination optical fibers, the detection optical fiber, and the sensing electrode are positioned on a probe.

15. The apparatus according to claim 11, wherein the plurality of illumination optical fibers transmits each source light in serial order.

16. The apparatus according to claim 15, wherein the detection optical fiber collects the returning light about every 10 milliseconds.

17. The apparatus according to claim 11, wherein the wavelength of the source light transmitted by each of the plurality of illumination optical fibers is about 690 nanometers or about 830 nanometers.

18. The apparatus according to claim 11, wherein the returning light has a returning intensity and the tissue spectrometer determines the returning intensity to provide readings of optical diffuse reflectance of the peripheral nerve after the electrical stimulation is applied to the peripheral nerve.

19. The apparatus according to claim 11, wherein the plurality of illumination optical fibers and the detection optical fiber are positioned on opposite sides of the peripheral nerve.

20. The apparatus according to claim 11, wherein the electrical nerve stimulator sends a synchronization signal to the tissue spectrometer when the electrical stimulation is applied to the peripheral nerve.

21. The apparatus according to claim 11 wherein the electrical stimulation from the electrical nerve stimulator is applied with a current below a threshold of visible tissue twitching.

22. The apparatus according to claim 11, wherein the electrical stimulation from the electrical nerve stimulator is applied with a current from 10 to 40 milliamperes.

23. The apparatus according to claim 11, wherein each of the plurality of separation distances is 1 to 4 centimeters.

24. A method for examining a peripheral nerve with an apparatus comprising an electrical nerve stimulator, a tissue spectrometer, an electrical stimulation probe, an illumination optical fiber; and a detection optical fiber, the method comprising:
applying, with the electrical stimulation probe, an electrical stimulation from the electrical nerve stimulator to a peripheral nerve;
transmitting, with the illumination optical fiber, a source light, having a wavelength in the near-infrared range, from the tissue spectrometer to the peripheral nerve;
collecting, with the detection optical fiber, a returning light from the peripheral nerve after the source light is transmitted to the peripheral nerve; and
delivering, with the detection optical fiber, the returning light to the tissue spectrometer.

25. The method according to claim 24, wherein the apparatus further comprises a sensing electrode, and wherein the method further comprises collecting, with the sensing electrode, electrical data from the peripheral nerve after the electrical stimulation is applied to the peripheral nerve.

26. A method of examining a peripheral nerve function and structure in a mammal comprising stimulating the said nerve and collecting the near infrared spectroscopy (NIRS) of the said nerve, wherein the point of NIRS data collection on said nerve is downstream of nerve impulse propagation from the point of stimulation.

27. The method of claim 26, wherein the nerve stimulation is performed by a nerve stimulator and the near infrared spectroscopy (NIRS) is performed by a tissue spectrometer.

28. The method of claim 26, further comprising detecting and measuring muscle activity of the muscle that innervated by the said peripheral nerve.

29. The method of claim 26, wherein the mammal is healthy or is afflicted with a disease.

30. The method of claim 29, wherein the disease is selected from a group consisting of metabolic disease, infectious disease, cancer, autoimmune disease, peripheral neuropathy neuralgia Bell's palsy; reflex sympathetic dystrophy syndrome; low-back pain; Guillain-Barré syndrome, and neuropathic pain.

31. The method of claim 26, wherein the mammal may be administered with a fluorescent chromophore or a fluorescent chromophore conjugate prior to peripheral nerve examination, wherein the said fluorescent chromophore or conjugate is taken up into nerves.
